(19) United States
(12) Patent Application Publication
(10) Pub. No.: US 2007/0036783 A1
(43) Pub. Date: Feb. 15, 2007

(54) ANTIBODY COMPLEXES

(75) Inventors: Laurent Humeau, Germantown, MD (US); Brian Paszkiet, Frederick, MD (US); Franck Lemiale, North Potomac, MD (US)

Correspondence Address: TOWNSEND AND TOWNSEND AND CREW, LLP, TWO EMBARCADERO CENTER, EIGHTH FLOOR, SAN FRANCISCO, CA, 94111-3834, US

(73) Assignee: VIRxSYS, Corporation, Gaithersburg, MD (US)

(21) Appl. No.: 11/424,637

(22) Filed: Jun. 16, 2006

Related U.S. Application Data
(60) Provisional application No. 60/691,631, filed on Jun. 16, 2005.

(30) Foreign Application Priority Data
Oct. 19, 2005 (JP) 2005-304670

(51) Int. Cl. A61K 39/395; C07K 16/28; C07K 16/46

(52) U.S. Cl. 424/143.1; 530/388.22; 530/391.1

(57) ABSTRACT

This invention is directed to a soluble complex of ligands that binds to surface molecules of hemopoietic cells and result in their activation or expansion. The complex may be used in the activation and/or expansion of hemopoietic cells, optionally in combination with their transduction. The complex of ligands bind at least two cell surface molecules, such as one that plays a role in cell-cell adhesion and one that may or may not activate or stimulate the cell to promote growth and/or proliferation after binding to a ligand. A complex of ligands that bind two hemopoietic cell stimulatory molecules is also provided. The invention further provides for the use of the complex to target vectors to hemopoietic cells.

### RELATED APPLICATIONS

This application claims benefit of priority under 35 U.S.C. § 119(e) from U.S. Provisional Patent Application 60/691,631, filed Jun. 16, 2005 and benefit of priority under 35 U.S.C. § 119 from Japanese Patent Application 2005-304670, filed Oct. 19, 2005, both of which are hereby incorporated in their entireties as if fully set forth.

### FIELD OF THE INVENTION

This invention relates to the activation, transduction, and/or expansion of hemopoietic cells, such as T cells. The invention provides for the use of a soluble complex of ligands that binds to surface molecules of such cells and result in their activation or expansion, optionally in combination with their transduction. Within the scope of the invention are complexes of ligands which bind at least two cell surface molecules, such as one that plays a role in cell-cell adhesion and one that may, or may not, activate or stimulate the cell to promote growth and/or proliferation after binding to a ligand. A complex of ligands that bind two hemopoietic cell stimulatory molecules may also be used. The invention further provides methods of using soluble complexes of ligands to activate hemopoietic cells.

### BACKGROUND OF THE INVENTION

Methods for the growth and propagation of T cells in vitro have been based upon a number of different approaches. In some, the T cells are maintained by use of accessory cells and exogenous growth factors, such as IL-2. One complication presented by the use of accessory cells is the need for MHC-matched antigen presenting cells (APCs), which are rather short lived, as the accessory cells. APCs must therefore be continually replenished into a T cell culture.

In others, a ligand, such as an anti-CD3 antibody, is used with a growth factor like IL-2 to stimulate T cell proliferation of the CD3+T cell subpopulation. One form of this approach is described in U.S. Pat. 6,352,694, where the anti-CD3 antibody, and an anti-CD28 antibody, are immobilized on a solid surface and then contacted with T cells.

Citation of documents herein is not intended as an admission that any thereof is pertinent prior art. All statements as to the date or representation as to the contents of documents is based on the information available to the applicants and does not constitute any admission as to the correctness of the dates or contents of these documents.

### SUMMARY OF THE INVENTION

The disclosed invention provides for the activation, transduction, and/or expansion of hemopoietic cells, such as T cells, based upon binding of ligands to cell surface molecules of said cells. The ligands are in the form of complexes able to bind at least two different cell surface molecules on the same cell. As such, the complexes are at least “bispecific”. In some embodiments, the ligands are antibodies or fragments thereof which bind the cell surface molecules. The complexes of the invention are soluble as opposed to being attached or otherwise immobilized on a solid support.

Thus in a first aspect, the invention provides complexes and compositions comprising cell surface binding ligands as described herein. In some embodiments, a complex of the invention is of at least two ligands wherein the first binds a cell surface molecule that plays a role in cell-cell adhesion and the second binds a cell surface molecule that activates or stimulates the cell into growth and/or proliferation after binding to the ligand(s) of a complex. In other embodiments, a complex of ligands that binds two cell stimulatory molecules may also be used. Activation or stimulation of a cell may be considered as an event followed by cell cycle transition out of a quiescent state, characterized, for example, by increased cellular size and/or alteration of cell surface marker expression patterns.

In addition to the ligands, a complex of the invention may comprise one or more linker molecules that are attached or otherwise bound to the ligands to keep them as part of the complex. The linker molecules may be any suitable chemical entity, including, but not limited to, one or more antibodies that bind the ligands. In embodiments wherein the ligands are antibodies, the linker molecules may be considered “secondary” antibodies that bind the constant region of “primary” antibodies that are the ligands. In other embodiments, the linker molecule may be a protein A or protein G derivative that binds the antibodies that are the ligands.

The complexes may be used to activate or expand cells in place of accessory cells or solid surface containing one or more cell binding ligands. The complexes also may be used to reduce the need for exogenous growth factors, such as cytokines. Additionally, and in cases of cells which recognize an antigen (such as T cells and B cells), the complexes can be used with cells in the absence of antigen as a stimulatory factor. This allows for the expansion of a treated cell population that is polyclonal in nature. In the case of treated T cells, this may be shown by the diversity of the T cell receptor (TCR) Vβ repertoire in cells of the treated population.

Thus in additional aspects of the invention, the complexes are used to activate and/or induce the cells into proliferation such that the total number of cells in the population increases. In other aspects, the complexes are used to stimulate the cells to grow such that cell size, volume and/or content increases. Thus the invention further provides methods of using soluble complexes of ligands to activate cells. Such activation includes inducing the cells to proliferate or expand, optionally in combination with genetic modification of the cells with a nucleic acid, such as with a viral vector.

Optionally, and in the case of T cells, induction of proliferation may be mediated by activating T cells via contact with an anti-CD3 antibody and stimulating an accessory molecule on the surface of the T cells with an anti-CD28 antibody, wherein the two antibodies are presented in a complex comprising them as ligands.

### BRIEF DESCRIPTION OF THE DRAWINGS

FIG. 1 is a schematic showing an embodiment contemplated for practice with the present invention.

FIG. 2 shows cell expansion levels after treatment with a tetrameric antibody complex (TAC). Legend: NV, no vector; 20D0/20D1, MOI 20 added on Day 0 and Day 1; 40D0, MOI 40 added on Day 0; 40D1, MOI 40 added on Day 1.

FIG. 3 shows measurements of cell size after treatment with TAC. Legend: NV, no vector; 20D0/20D1, MOI 20 added on Day 0 and Day 1; 40D0, MOI 40 added on Day 0; 40D1, MOI 40 added on Day 1.

FIG. 4 shows the levels of transduction assessed by eGFP, plus CD25 and CD69 expression in cells treated with TAC. Legend: NV, no vector; 20D0/20D1, MOI 20 added on Day 0 and Day 1; 40D0, MOI 40 added on Day 0; 40D1, MOI 40 added on Day 1.

FIG. 5 shows the results of QPCR analysis to detect vector transduction efficiency. Legend: NV, no vector; 20D0/20D 1, MOI 20 added on Day 0 and Day 1; 40D0, MOI 40 added on Day 0; 40D1, MOI 40 added on Day 1.

FIG. 6 shows the results of an analysis of TCR Vβ repertoire in cells treated with TAC. Legend: CV C, Control Vector, CD3 soluble; NV C, No Vector, CD3 soluble; CV T, Control vector, TAC; NV T, No Vector, Tac.

FIG. 7 shows an analysis of cell viability after TAC treatment. Legend: CV C, Control Vector, CD3 soluble; NV C, No Vector, CD3 soluble; CV T, Control vector, TAC; NV T, No Vector, Tac FIG. 8 shows results from an analysis of cytokine production in cells treated with TAC and stimulated with superantigen Staphylococcus Enterotoxin B (SEB). Legend: CV C, Control Vector, CD3 soluble; NV C, No Vector, CD3 soluble; CV T, Control vector, TAC; NV T, No Vector, Tac FIG. 9 shows the efficiency of vector transduction, as measured by percent GFP expression at day 7 in culture of T cells that were activated and expanded with TAC. T cells were isolated from a panel of 4 HIV patients with the following viral loads (vl) and CD4 counts: 001-058-Z, vl=75 copies/ml, CD4=1200 cells/μl; 001-059-G, vl=45,176 copies/ml, CD4=290 cells/μl; 001-062-J, vl=301 copies/ml, CD4=720 cells/μl; and 001-063-K, vl=10,132 copies/ml, CD4=610 cells/μl.

FIG. 10 shows the efficiency of vector transduction as measured by number of vector copies per cell in T cells that were activated and expanded with TAC, from the panel of 4 HIV patients as described in the FIG. 9 legend above.

FIG. 11 reports the activation of T cells that were activated and expanded by TAC, from the panel of 4 HIV patients as described in the FIG. 9 legend above. Cells were measured for cell size using a Z2 Coulter counter. Non-transduced (no vector or NV) matched controls are also shown.

FIG. 12 shows the expansion profiles of the T cells from the panel of 4 HIV patients described in the FIG. 9 legend above. Non-transduced (no vector or NV) matched controls are also shown. FIG. 13 demonstrates the biological activity of the anti-HIV vector in the panel of 4 HIV patients described in the FIG. 9 legend above. The effect of the vector against production of p24, a surrogate marker for HIV replication, is shown. Matched non transduced (no vector or NV) controls are shown for each patient. The p24 values in the culture for patient 63 is shown for the whole time course as an example.

### DETAILED DESCRIPTION OF MODES OF PRACTICING THE INVENTION

This invention is based upon the use of soluble complexes of ligands which bind cell surface molecules of a target hemopoietic (or hematopoietic) cell. Hemopoietic cells are those found in blood or other in vivo locations, such as, but not limited to, bone marrow. Target hemopoietic cells for use in the practice of the invention include leukocytes, including lymphocytes (T cells and B cells), monocytes, and granulocytes (eosinophils, basophils, and neutrophils), and erythrocytes. In much of the disclosure herein, T cells are used as a non-limiting example of a hemopoietic cell in the practice of the invention. Other hemopoietic cells may be used in a similar fashion, with cell type specific changes made as needed based on the knowledge of the skilled person in the field.

As used herein, “cell surface molecule” refers to a molecule present on the surface of a cell, such as a hemopoietic cell. The molecule may be a single molecule or a complex of a plurality of molecules. The term includes proteinaceous molecules that contain a portion that is transmembrane in nature as well as molecules that are associated with the cell surface, such as by interactions with a transmembrane molecule, without limitation. The molecules may be polypeptides that are modified, such as by glycosylation, phosphorylation or acylation as non-limiting examples. Other non-limiting examples of molecules include cell surface markers. In some embodiments of the disclosed invention, such as with T cells, one ligand may bind an antigen specific cell surface molecule of a cell (like an anti-CD3 antibody as the ligand) and the other ligand may bind a non-antigen specific cell surface molecule of a cell (like an anti-CD28 antibody as the ligand).

In embodiments with T cells as the target, the ligands may bind any molecule found on the surface of T cells. Non-limiting examples of cell surface molecules include B7-H1 (also called PD-L1); B7-H2; B7-H3 (also called B7RP-2); B7-H4; CD2; CD3 or CD3/TCR complex; CD11a; CD26; CD27; CD28; CD30L; CD32; CD38; CD40L (also called CD154); CD45; CD49; CD50 (also called ICAM-3); CD54 (also called ICAM-1); CD58 (also called LFA-3); CD70; CD80 (also called B7.1); CD86 (also called B7.2); CD100; CD122; CD137L (also called 4-1 BB Ligand); CD153; CTLA-4 (also called CD152); ICOS; OX40L (also called CD134); PD-1; PD-L2 (also called B7-DC); SLAM (also called CD150); TIM-1; TIM-2; TIM-3; TIM-4; and 2B4 (also called CD244). In other non-limiting embodiments, the cell surface molecule is CD28, CD7, ICOS-L, ICAM, CD40, CD83, HLA-G, MICA, MICB, HVEM, lymphotoxin beta receptor, ILT3, ILT4, 3/TR6, 4-1BB, OX40, CD30, CD40, ICOS, LFA-1, CD7, LIGHT, NKG2C, BTLA, a Toll ligand receptor, or CD83. In further embodiments, the ligand may be one that specifically binds to B7-H3 or CD83.

In some embodiments, the invention is practiced with complexes with ligands that bind each of the above described molecules. Stated differently, a complex of the invention comprises ligands which bind cell surface molecules selected from those listed above, without limitation as to the combination of ligands (and thus combination of cell surface molecules bound by a complex of the invention. Combinations of more than one ligand (such as antibodies) that bind the same cell surface molecule may also be used in complexes of the invention. Where the ligands of a complex bind the same cell surface molecule, the ligands may be the same or different. Thus, as a non-limiting example, if the complex comprised two ligands that bind CD28, the ligands may be two identical monoclonal antibodies that bind CD28 or two different monoclonal antibodies that both bind CD28.

Continuing with CD28 as a non-limiting example, a monoclonal antibody or fragment thereof capable of binding or crosslinking the CD28 molecule, or a natural ligand for CD28 (such as a protein in the B7 family like B7-1(CD80) and B7-2(CD86)) can be used in the practice of the invention. In some embodiments, a complex comprises a first ligand which binds CD28. The second ligand may bind another cell surface molecule, such as those listed in the above paragraph. One non-limiting example is where the second ligand binds the CD3/TCR complex and/or the CD2 molecule of a T cell. Where the first ligand binds CD28, embodiments of the invention include a complex comprising a second ligand which binds CD3 or the CD3/TCR complex. Each of a first and second ligand in a complex may be an antibody or a fragment thereof which binds a first cell surface molecule and a second cell surface molecule, respectively. Non-limiting examples of such a complex include a tetrameric antibody complex (TAC) comprising two antibodies that act as the first and second ligands that are linked by two linker antibodies that bind the antibodies acting as first and second ligands. The linker antibodies may conveniently bind the constant region of antibodies, and where the constant regions are of different isotypes, a bi-specific antibody with one binding region for each isotype may also be used.

In other embodiments, the antibodies, or antigen binding fragments thereof, that act as first and second ligands may be covalently or non-covalently bound by one or more linker molecules. Non-limiting examples of such linker molecules include avidin or strepavidin, which may be used to join biotinylated antibodies, such as antibodies with biotin moieties in the Fc region. In additional embodiments, tetrameric antibody complexes may be used as a mixture of complexes. This includes use of more than one species of complex in a mixture of complexes, wherein the complexes of the entire mixture can contact more than 2 different ligands. As a non-limiting example, a given mixture could contain a first tetrameric antibody complex which targets (binds) a first and second ligand, and a second tetrameric antibody complex which targets (binds) a third and forth ligand.

In another aspect, the invention provides a method for activating a hemopoietic cell, such as a T cell, the method comprising contacting the cell with a complex of the invention. The method may be used to activate the cell to proliferate or expand. Activation may also be to induce the cell to increase production of one or more gene products or cellular factors. Alternatively, a method of stimulating a hemopoietic cell to grow is provided by similarly contacting the cell with a complex as described herein. The contacting may be performed in vitro or with hemopoietic cells (like T cells) that are ex vivo, such as those obtained from a human or animal subject's peripheral blood or as part of the peripheral blood mononuclear cell (PBMC) fraction. Alternatively, the contacting may be in vivo, such as by administration of a complex to a human or animal subject.

The invention is based in part on the discovery of the complexes as suitable for introduction into a human or animal subject. Given that the complexes bind and activate endogenous cells, it has been unpredictable as to the results of their introduction into a human or animal subject. This was irrespective of whether they complexes are administered directly or in a cell associated form after their contact with cells treated ex vivo. It has also been unpredictable as to whether ex vivo (or in vitro) culturing of treated cells, or alternatively removal of the complexes by washes or changes of medium, would be sufficient to render cells treated with complexes suitable for introduction into a human or animal subject.

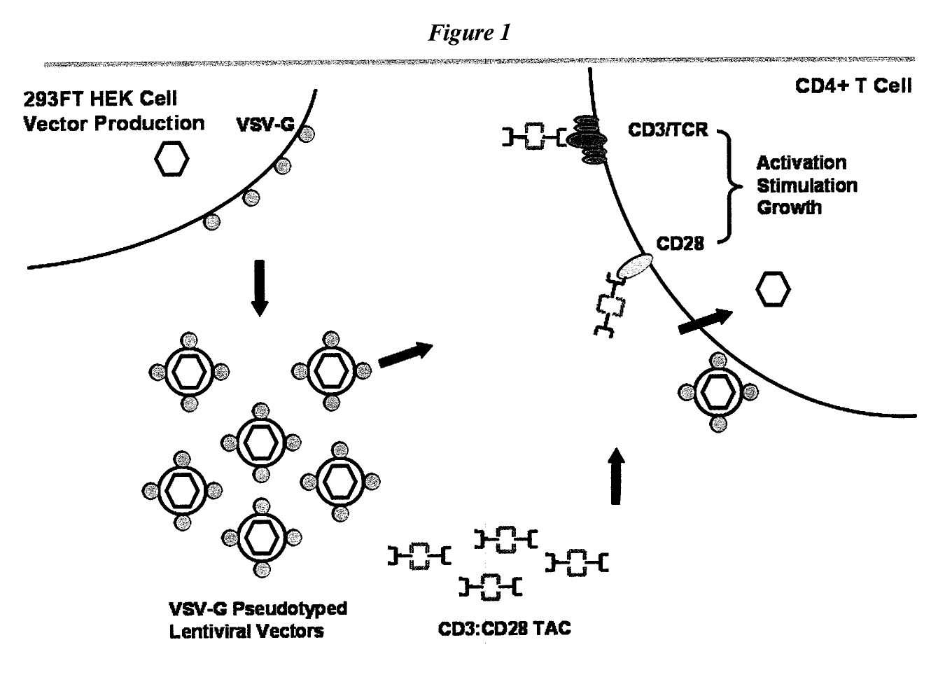

Additional embodiments of the invention include those wherein a complex is used in combination with a vector to increase the transduction efficiency of the vector. FIG. 1 shows a non-limiting schematic to illustrate this embodiment. Vectors are shown as having been produced by a 293 HEK (human embryonic kidney) cell which pseudotypes a lentiviral vector with a VSV-G (vesicular stomatitis virus G) envelope protein. The packaged vectors are used to transduce CD4+ T cells in the presence of a TAC containing an antibody which binds CD3/TCR and an antibody which binds CD28. The TAC results in the activation, stimulation, and/or growth of the T cell during transduction with the vector.

Thus one non-limiting example of the invention provides for T cell stimulation comprising the use of an anti-CD3/anti-CD28 TAC. In the case of anti-human CD3 and anti-human CD28 antibodies that are both murine in origin, rat anti-mouse IgG1 antibodies (as well as other anti-mouse antibodies, such as, but not limited to, those from goat, sheep, horse, rabbit, human, monkey, hamster, or pig) may be used to form TAC with the mouse antibodies. Cells were cultured with and without the TAC, transduced with an eGFP-expressing lentiviral vector, and were tested as described in the Examples below. The invention may also be practiced with the use of anti-mouse antibodies that have been humanized as a non-limiting alternative.

In some embodiments of the invention, the antibodies, or CD3 and CD28 binding fragments thereof, are human or humanized antibodies rather than those of a non-human species. The human or humanized antibodies are then bound by “secondary” antibodies that recognize the human or humanized region of the anti-CD3 and anti-CD28. In some embodiments, these secondary antibodies are humanized antibodies comprising a non-human binding region. The use of human or humanized antibodies may be advantageously applied to human cells, or human subjects in vivo, to reduce or minimize immune responses against a non-human or non-humanized antibody.

Where the invention is practiced in culture, the cells may be used in the presence of a growth factor, such as, but not limited to, IL-2 or a combination of IL-7 and IL-15. In additional embodiments, other T cell specific cytokines or chemokines may be used. Non-limiting examples include IL-4, MIP 1 alpha (macrophage inflammatory protein 1-alpha), MIP 1 beta (macrophage inflammatory protein 1-beta), and RANTES (regulated on activation, normal T cell expressed and secreted).

Use of the above described TAC was also observed to provide multiple unexpected results on T cells. The results include 1) higher viability and 2) greater cytokine response to stimulation with antigen in TAC treated cells than in cells treated with the same antibodies attached to a solid surface. Additional advantages of the invention include significant expansion of activated cells; preservation of T cell diversity (i.e. clonal diversity) in the expanded cells; and >90% transduction efficiencies.

In addition to the illustration of a simultaneous contacting of a cell with a vector, or other nucleic acid, while the cell is contacted with a complex of the invention, the invention provides other methods of transducing a hemopoietic cell. Such methods comprise introducing a nucleic acid molecule into said cell before or after said cell is contacted with a complex of the invention. Transduction methods of the invention are preferably performed in vitro or ex vivo, such as with T cells obtained from a human or animal subject's peripheral blood or as part of the peripheral blood mononuclear cell (PBMC) fraction. Of course such cells may be returned to a subject after ex vivo treatment. Alternatively, the contacting may be in vivo, such as by administration of a vector or other nucleic acid, in combination with a complex of the invention, to a human or animal subject.

While the invention has been predominantly described in the context of CD4+ T cells, it may also be used in relation to other hemopoietic cell (or T cell) populations. As one non-limiting example, the methods of the invention may be used to expand different populations of T cells. Non-limiting examples beyond CD4+ cells include CD28+, CD8+ T cells and CD3+ T cells. Other non-limiting examples include cells expressing any of the cell surface molecules described herein. The invention may also be applied to the expansion of antigen specific cells. The resultant cells can be used in a variety of clinical and research uses, including, but not limited to, the treatment of infections, infectious diseases, genetic disorders, and cancer. In some embodiments, the invention can produce T cells which are polyclonal with respect to antigen reactivity, but either homogeneous or heterogeneous with respect to certain cell surface markers. The resultant T cells may also be transduced and used for immunotherapy.

Various methods may be used to transduce hemopoietic cells, such as T cells. In some embodiments of the invention, a cell is transduced with a vector or plasmid. The term “vector” or “plasmid” refers to a nucleic acid molecule capable of transporting a nucleic acid sequence between different cellular or genetic environments. Different cellular environments include different cell types of the same organism while different genetic environments include cells of different organisms or other situations of cells with different genetic material and/or genomes. Non-limiting vectors of the invention include those capable of autonomous replication and expression of nucleic acid sequences (or “payload”) present therein. Vectors may also be inducible for expression in a way that is responsive to factors specific for a cell type. Non-limiting examples include inducible by addition of an exogenous modulator in vitro or systemic delivery of vector inducing drugs in vivo. Vectors may also optionally comprise selectable markers that are compatible with the cellular system used. One type of vector for use in the practice of the invention is maintained as an episome, which is a nucleic acid capable of extra-chromosomal replication. Another type is a vector which is stably integrated into the genome of the cell in which it is introduced.

The types of vectors used for transduction include those based upon any virus. Vectors derived from retroviruses, including avian reticuloendotheliosis virus (duck infectious anaemia virus, spleen necrosis virus, Twiehaus-strain reticuloendotheliosis virus, C-type retrovirus, reticuloendotheliosis virus Hungary-2 (REV-H-2)), and feline leukemia virus (FeLV)), are particular non-limiting examples. Retroviral genomes have been modified for use as a vector (Cone & Mulligan, Proc. Natl. Acad. Sci., USA, 81:6349-6353, (1984)). Non-limiting examples of retroviruses which may be used as vectors of the invention include lentiviruses, such as human immunodeficiency viruses (HIV-1 and HIV-2), feline immunodeficiency virus (FIV), simian immunodeficiency virus (SIV), Maedi/Visna virus, caprine arthritis/encephalitis virus, equine infectious anaemia virus (EIAV), and bovine immunodeficiency virus (BIV); avian type C retroviruses, such as the avian leukosis virus (ALV); HTLV-BLV retroviruses, such as bovine leukaemia virus (BLV), human T-cell lymphotropic virus (HTLV), and simian T-cell lymphotropic virus; mammalian type B retroviruses, such as the mouse mammary tumor virus (MMTV); mammalian type C retroviruses, such as the murine leukaemia virus (MLV), feline sarcoma virus (FeSV), murine sarcoma virus, Gibbon ape leukemia virus, guinea pig type C virus, porcine type C virus, wooly monkey sarcoma virus, and viper retrovirus; spumavirus (foamy virus group), such as human spumavirus (HSRV), feline synctium-forming virus (FeSFV), human foamy virus, simian foamy virus, and bovine syncytial virus; and type D retroviruses, such as Mason-Pfizer monkey virus (MPMV), squirrel monkey retrovirus, and langur monkey virus.

Lentiviral and retroviral vectors may be packaged using their native envelope proteins, or may be modified to be encapsulated with heterologous envelope proteins. Examples of envelope proteins include, but are not limited to, an amphotropic envelope, an ecotropic envelope, or a xenotropic envelope, or may be an envelope including amphotropic and ecotropic portions. The protein also may be that of any of the above mentioned retroviruses and lentiviruses. Alternatively, the env proteins may be modified, synthetic or chimeric env constructs, or may be obtained from non-retroviruses, such as vesicular stomatitis virus and HVJ virus. Specific non-limiting examples include the envelope of Moloney Murine Leukemia Virus (MMLV), Rous Sarcoma Virus, Baculovirus, Jaagsiekte Sheep Retrovirus (JSRV) envelope protein, and the feline endogenous virus RD114; gibbon ape leukemia virus (GALV) envelope; baboon endogenous virus (BaEV) envelope; simian sarcoma associated virus (SSAV) envelope; amphotropic murine leukemia virus (MLV-A) envelope; human immunodeficiency virus envelope; avian leukosis virus envelope; the endogenous xenotropic NZB viral envelopes; and envelopes of the paramyxoviridiae family such as, but not limited to the HVJ virus envelope.

The vectors of the invention may include genetic material encoding a “payload” which is expressed in the packaging cell and/or the target cell after delivery of the vector to a target cell. The fact that the vectors are packaged into a particle also permits the “payload”, or a biological product that is produced upon expression of the genetic material encoding the “payload”, to be incorporated into the packaged particle for delivery to a target cell. One “payload” of the invention is a therapeutic agent that is encoded by the vector's genome. Of course a “payload” that is a polypeptide or a nucleic acid (such as RNA) may also be expressed in the packaging cell and physically present in the packaged particle in addition to, or instead of, being expressed in the target cell. In some embodiments, a “payload” is not toxic or is minimally toxic to the packaging cell used.

Non-limiting examples of genetic material encoding a therapeutic agent include polynucleotides encoding tumor necrosis factor (TNF) genes, such as TNF-α; genes encoding interferons such as Interferon-α, Interferon-β, and Interferon-γ; genes encoding interleukins such as IL-1, IL-1β, and Interleukins 2 through 14; genes encoding GM-CSF; genes encoding adenosine deaminase, or ADA; genes which encode cellular growth factors, such as lymphokines, which are growth factors for lymphocytes; genes encoding antibodies; genes encoding apoptotic or pro-death genes, such as tumor necrosis factor related apoptosis inducing ligand (TRAIL); gene encoding products that inhibit angiogenesis; genes encoding epidermal growth factor (EGF), and keratinocyte growth factor (KGF); genes encoding soluble CD4; Factor VIII; Factor IX; cytochrome b; glucocerebrosidase; T-cell receptors; the LDL receptor, ApoE, ApoC, ApoAl and other genes involved in cholesterol transport and metabolism; the alpha-1 antitrypsin (α1AT) gene; the insulin gene; the hypoxanthine phosphoribosyl transferase gene; the CFTR gene; genes allowing for positive selection of cells, such as mutant methyl guanine transferase (MGMT); negative selective markers or “suicide” genes, such as viral thymidine kinase genes, such as the Herpes Simplex Virus thymidine kinase gene, the cytomegalovirus virus thymidine kinase gene, and the varicella-zoster virus thymidine kinase gene; Fc receptors for antigen-binding domains of antibodies, antisense sequences which inhibit viral replication, such as antisense sequences which inhibit replication of hepatitis B or hepatitis non-A non-B virus; antisense c-myb oligonucleotides; and genes encoding antioxidants such as, but not limited to, manganese superoxide dismutase (Mn-SOD), catalase, copper-zinc-superoxide dismutase (CuZn-SOD), extracellular superoxide dismutase (EC-SOD), and glutathione reductase; the multidrug resistance (MDR) gene; polynucleotides encoding ribozymes, antisense polynucleotides; polynucleotides encoding micro RNAs and hairpin RNAs for induction of RNAi (RNA interference ) such as by use of siRNA (short interfering RNA), aptamers, and/or RNA decoys; genes encoding secretory peptides which act as competitive inhibitors of angiotensin converting enzyme, of vascular smooth muscle calcium channels, or of adrenergic receptors; genes encoding zinc finger nucleases; and polynucleotides encoding enzymes which break down amyloid plaques within the central nervous system.

Genetic material encoding the following may also be used: tissue plasminogen activator (tPA); urinary plasminogen activator (urokinase); hirudin; the phenylalanine hydroxylase gene; nitric oxide synthase (endothelial and neuronal); vasoactive peptides; angiogenic and anti-angiogenic peptides; the dopamine gene; the dystrophin gene; the β-globin gene; the α-globin gene; the HbA gene; protooncogenes such as the ras, src, and bcl genes; tumor-suppressor genes such as p53 and Rb; the LDL receptor; the heregulin-αprotein gene, for treating breast, ovarian, gastric and endometrial cancers; and monoclonal antibodies specific to epitopes contained within the β-chain of a T-cell antigen receptor. It is to be understood, however, that the scope of the present invention is not to be limited to any particular therapeutic agent.

In some embodiments of the invention, the payload can be a gene encoding a clotting factor (e.g., Factor VIII or Factor IX) useful in the treatment of hemophilia, or the gene can encode one or more products having another therapeutic effect. Examples of suitable genes include those that encode cytokines such as TNF, interleukins (interleukins 1-12), interferons (α, β, and γ-interferons), T-cell receptor proteins and Fc receptors for binding to antibodies.

The vectors of the invention are useful in the treatment of a variety of diseases including but not limited to infectious diseases such as viral infections like HIV (Human Immunodeficiency Virus), HCV (Hepatitis C Virus), and herpes infections; genetic based disorders such as cancer, adenosine deaminase deficiency, sickle cell anemia, thalassemia, hemophilia, diabetes, α-antitrypsin deficiency, brain disorders such as Alzheimer's disease or Parkinson's disease; and other illnesses such as growth disorders and heart diseases, for example, those caused by alterations in the way cholesterol is metabolized and defects of the immune system.

In other embodiments, the vectors of the invention may include a negative selectable marker, such as, for example, a viral thymidine kinase gene, and more particularly, the Herpes Simplex Virus thymidine kinase (TK) gene. Such vectors may be administered to cancer cells (in particular to tumor cells) in a human patient in vivo or used ex vivo. The vectors then transduce the tumor cells. After the vectors have transduced the tumor cells, the patient is given an interaction agent, such as gancyclovir or acyclovir, which interacts with the protein expressed by the negative selectable marker in order to kill all replicating cells (i.e., the cancer or tumor cells) which were transduced with the vector including the negative selectable marker. Alternatively, vectors may be used to transduce progenitor or stem cells of any origin, for expression of genes encoding cell surface pro-death or apoptosis inducing factors, such as the TRAIL ligand, that are specific to tumors. It has been established that such progenitor or stem cells are capable of trafficking to sites of tumors, and therefore would contact tumors with the pro-death gene to mediate a therapeutic effect.

The vectors of the invention may also encode molecules known as “molecular decoys”. Such “decoys” may be binding elements for viral proteins needed for viral replication or assembly, such as TAR. The vectors can also be capable of expressing antisense molecules and ribozymes directed against particular nucleic acid molecules, such as those which are expressed to permit viral replication or infection.

The invention may also be applied to the in vivo expansion of hemopoietic cells, such as CD4+ T cells, in a subject, such as a human being. The administration of a complex of the invention is generally a form of protein therapy to activate or stimulate T cells in vivo. This may be advantageously used in cases of an HIV infected or other immunodeficient individual. Alternatively, T cells from a subject may be treated by the methods of the invention in vitro or ex vivo and then returned to the individual.

In additional embodiments, methods of the invention may be used to selectively expand a subpopulation of cells within a mixed or otherwise heterogeneous population of cells. Non-limiting examples include the expansion of CD4+ or CD8+ cells in a mixed population of lymphocytes. The cells to be expanded may also optionally be selected based on their specificity for an antigen, such as by stimulation with the antigen. In other embodiments, methods of the invention may be directed in their use to Th1 or Th2 cells such that they are activated, transduced, and/or expanded. In other embodiments, the cells may be cells with a memory or naive phenotype; pheripheral or central cells; or simply Th cells without limitation to Th1 or Th2.

Antibodies of the Invention and Compositions Comprising them

The invention provides antibody compositions for use in the complexes and methods described herein. The antibody compositions contain at least two antibodies that bind to two different cell surface molecules (or antigens from the perspective of the antibodies) on a hemopoietic cell, such as a T cell. In some embodiments, the antibodies that bind the cell surface molecules are human or humanized antibodies rather than non-human, such as mouse, antibodies. In some embodiments, the antibodies may be humanized mouse antibodies. The term “at least two antibodies” refers to the antibody composition including at least two types of antibodies (as opposed to at least one type of antibody molecule which binds to one cognate antigen). As a non-limiting example, antibodies that bind to the CD3 antigen are considered one type of antibody.

In some embodiments, the two antibodies (1) and (2) may be linked directly to form bifunctional antibodies. Chemical coupling may be used to form bifunctional antibodies. A non-limiting example is the chemically coupling of one antibody to another by using N-succinimidyl-3-(2-pyridyldithio) propionate (SPDP).

Alternatively, the two antibodies (1) and (2) may be indirectly linked or a TAC may be formed as described herein. Non-limiting examples include use of bispecific antibodies containing a variable region specific for antibody (1) and a variable region specific for antibody (2). Non-limiting examples include bispecific antibodies with variable regions that bind the constant regions of antibodies (1) and (2) where they are different. Hybrid hybridomas may be used to generate bispecific antibodies. See Staerz & Bevan, (1986, PNAS (USA) 83: 1453) and Staerz & Bevan, (1986, Immunology Today, 7:241) for exemplary procedures known to the skilled person. Chemical means may also be used to construct bispecific antibodies; see Staerz et al., (1985, Nature, 314:628) and Perez et al., (1985 Nature 316:354).

Other chemical conjugation based means include those using homo- and heterobifunctional reagents with E-amino groups or hinge region thiol groups. Non-limiting homobifunctional reagents such as 5,5′-dithiobis(2-nitrobenzoic acid) or DNTB generate disulfide bonds between two Fabs. Alternatively, 0-phenylenedimaleimide (O-PDM) can be used to produce thioether bonds between two Fabs. A heterobifunctional reagent like SPDP combines exposed amino groups of antibodies and Fab fragments, regardless of class or isotype.

Other means to produce bispecific antibodies include expression of recombinant immunoglobulins. Recombinant DNA based technology is used to combine DNA sequences encoding antibody fragments into nucleic acid constructs which can be used to express the recombinant protein. Alternatively, bispecific antibodies can be produced as a single covalent structure via linker mediated combination of two single chain Fv (scFv) fragments.

Bifunctional antibodies also may be generated by somatic hybridization, where fusion of two established hybridomas creates a quadroma; or fusion of one established hybridoma with lymphocytes derived from an animal or human (and which bind a second antigen) to create a trioma.

Indirect linkage of antibodies (1) and (2) refers to the antibodies not being directly covalently linked to each other. Instead, they are attached through a linking molecule such as an immunoglobulin that binds both. Where two such immunoglobulins are used, a TAC is the result. Such a TAC may be prepared by mixing a first monoclonal antibody (1) and a second monoclonal antibody (2) from the same first animal species. Alternatively, antibodies (1) and (2) may be human or humanized antibodies. Antibodies (1) and (2) are then contacted with an equimolar (or about equimolar) amount of antibodies (optionally monoclonal) of a second animal species which bind the Fc portions of antibodies (1) and (2). As an additional alternative, antibodies (1) and (2) are reacted with an equimolar (or approximately equimolar) amount of an F(ab′)₂ fragment of antibodies (optionally monoclonal) where the fragment binds the Fc portions of antibodies (1) and (2).

As used herein, antibodies may be monoclonal antibodies or polyclonal antibodies. The invention also contemplates the use of antibody fragments (such as Fab and F(ab′)₂), chimeric antibodies, and bifunctional or bispecific antibodies in the formation of TACs. An antibody is considered to bind, or be reactive against, a cell surface molecule (or antigen) if the antibody (or antigen binding fragment thereof) binds non-randomly, such as with a sufficient dissociation constant to not bind other molecules present (naturally or by human intervention) with the surface molecule. Binding specificity may be determined based on the ability of the antibody to differentially bind a cell surface molecule and an unrelated antigen so as to distinguish between two different antigens. This is readily understood with respect to antibodies that bind epitopes unique to individual antigens. A “specific” antibody binds specifically to a particular epitope relative to other epitopes.

Fragments of antibodies can be produced by techniques known to the skilled person, and the fragments screened for binding to a cell surface molecule. As non-limiting examples, F(ab′)₂ fragments can be generated by pepsin digestion, optionally followed by reducing conditions to produce Fab′ fragments.

Chimeric antibodies are antibody molecules that combine a variable region and a constant region from different species. The term includes humanized antibodies, where a non-human animal variable region (such as one from mouse, rat or other species) is combined with a human constant region. TACs comprising humanized antibodies may be used in in vivo methods of the invention to increase their acceptance in a human subject.

In some embodiments, monoclonal antibodies (MAbs) are used in the practice of the invention. MAbs that bind cell surface molecules as described herein may be readily prepared by use of techniques known in the field. One non-limiting example is the use of the hybridoma or other technique known to the skilled person. The hybridoma cells can be screened for expression of antibodies that are specifically reactive with a cell surface molecule. MAbs may also be isolated before use.

TAC for Ex Vivo Cell Processing

This invention may be applied in the context of ex vivo expansion of cells, such as in the preparation of a medicament as a non-limiting example. To date, numerous clinical trials have been conducted using autologous (self) lymphocytes that have been removed from the body and expanded multiple times outside the body, in order to augment the number of cells available for treatment and improve therapeutic outcomes. Expansion of cells is limited by the efficiency of the method for stimulation of the cells ex vivo. For example, expansion via activation of T cells using cytokines or antigen presenting cells (APCs) has been successful but only allows for limited amounts of expansion. An alternative approach has been to use artificial APCs, which can be immobilized antibodies that bind to and cross-link the cognate APC receptors on the T cells, and this has historically shown higher levels of expansion.

The present invention offers an alternative for efficient expansion of cells. In addition, expansion using this technology has several unexpected benefits including greater viability, better preservation of immune potential of the expanded cells and preservation of T cell diversity while still enabling expansion and transduction of cells. Such benefits may be applicable to improve success in the clinic when using cellular products expanded by the invention. Potential applications of the instant invention include adoptive immunotherapy for cancer or HIV, as non-limiting examples.

As a non-limiting example, use of this invention for ex vivo cellular expansion may include the following steps: isolation of cells from a patient (such as by apheresis), optionally followed by purification of a cellular subset; contacting the cells with a TAC complex of the invention to induce activation and expansion, optionally performed concomitantly with genetic modification of the cells (such as by transduction with a vector); culturing the cells in the presence of the complexes for a period of time to allow for expansion; and either harvesting and freezing of the cells or immediate use of the cells.

These aspects of the invention may be summarized as a method for ex vivo expansion of cells for the preparation of a medicament, the method involving cell isolation and expansion using a complex of the invention. Stated differently, the invention provides a method for ex vivo expansion of cells in the preparation of a medicament, said method comprising cell isolation and expansion after contact with a complex of the invention. In such methods, the cells may be genetically modified as described herein, such as by use of a retroviral vector as a non-limiting example.

TAC Mediated Targeting of Vectors

The invention also provides for the use of TAC to target vectors to particular hemopoietic cell types. It is well known in the field that direct or specific targeting of any type of vector, including viral vectors and vectors described above, to a target cell in a mixed population of cells is a significant challenge. With viral vectors, for example, a major focus over the years has been to develop cell-specific envelopes for engineering into viral vectors. The envelopes of the viral particles thus target the desired cell type(s). However, progress in this area has been slow, and even with successful targeting in vitro, the engineered vectors often lose their ability to be purified for clinical application. This inability for successful purification stems from the inherent instability of many envelope proteins, which are then stripped or denatured to an extent during purification for clinical application, resulting in loss of viral vector titer and hence utility of the vector particle.

The invention may be used as an alternative to envelope engineering. As a non-limiting example, TAC can be used in a way that links a protein present on the membrane surface of the vector producer cell, to a protein present on and specific to the target cell of interest. Thus TACs may be combined with viral vectors (with the membrane surface protein of the producer cell) to form vector-TAC complexes specific for the targeted cell type. The combination of TAC and viral vectors is performed post vector production, such as at the time of use or injection in some embodiments of the invention, like those to improve stability or maintain sterility of the vector. For in vitro applications, non-human antibodies can be used for targeting purposes. For in vivo application, a preferred embodiment would be to use humanized antibodies in the TAC, such that there would be minimal increase in the immunogenicity of the targeted vectors.

Thus the invention further provides a method of directing an enveloped vector to a target hemopoietic cell. The method may comprise contacting the target cell with a combination of an enveloped vector and a complex of ligands, which bind cell surface molecules as described herein, to form a vector-complex combination. The complex comprises a first ligand which binds a cell surface molecule in the envelope of said vector and a second ligand which binds a cell surface molecule of the target cell. Any enveloped vector, or vector that may be enveloped, as known to the skilled person may be used in the practice of the method. Alternatively, the method comprises use of a combination of vector particle and complex, wherein the vector particle is not enveloped and the complex comprises a first ligand which binds the particle and a second ligand which binds a cell surface molecule of the target cell. A vector particle is a vector which is encapsulated, such as by a capsid. As would be understood by the skilled person, the first ligand may bind an exposed molecule or epitope of the vector particle.

Kit Embodiments of the Invention

This invention also provides compositions and kits comprising a complex of the invention. Such kits optionally further comprise an identifying description or label or instructions relating to the use of the kits, or the suitability of the kits, in the methods of the present invention. Such a kit may comprise containers, each with one or more of the various agents and/or reagents (optionally in concentrated form) utilized in the methods, including, for example, cell growth media and growth factors as needed or desired. A set of instructions or reagent identifiers will also typically be included. For example, a kit can comprise a composition of a soluble complex with an anti-CD3 antibody coupled to an anti-CD28 antibody. The composition may be lyophilized to aid storage.

Having now generally described the invention, the same will be more readily understood through reference to the following examples which are provided by way of illustration, and are not intended to be limiting of the present invention, unless specified.

### EXAMPLES

Example 1

Procedures

Peripheral blood mononuclear cells (PBMCs) were isolated from apheresis product by Ficoll™ density gradient separation. The PBMCs were CD4+ enriched using MACS® columns. The cells were frozen for later use.

Frozen CD4+ T-cells were thawed and cultured as follows. Cells were cultured in X-Vivo 15 medium and were then treated with the tetrameric antibody complex (TAC)).

The TAC consisted of 2 rat anti-mouse IgG1 antibodies bound to one mouse anti-human CD3 and one mouse anti-human CD28. The TAC is a mixture containing individual complexes with 2 anti-CD3, 2 anti-CD28, and 1 anti-CD3 with 1 anti-CD28 antibodies bound to 2 rat anti-mouse antibodies.

Cells were pre-incubated with TAC and then transduced with VRX494 vector.

The VRX494 vector is an eGFP expressing lentiviral vector. The different transductions may be summarized as follows:
Mock transduced—no vector (NV)
Transduced with MOI of 40 over the first two days of culture (20D0,20D1)
Transduced with MOI of 40 on the first day of culture (40D0)
Transduced with MOI of 40 on the second day of culture (40D1) On Day 7, cells were counted and sized, and analyzed for eGFP (a transduction marker), CD25 and CD69 expression (T-cell activation markers) using FACS (Fluorescence Activated Cell Sorting).

The various analysis performed may be summarized as follows:
Cell expansion levels and size were determined throughout 21 days
Cell transduction levels (eGFP) were analyzed on Day 7
   FACS analysis
   Quantitative PCR analysis (Taqman)
Cell activation marker (CD25 or CD69) expression was analyzed on Day 7
TCR Vβ marker expressions were determined on Day 14
Intracellular cytokine staining was performed on Day 14
Cell viability was determined by FACS on Day 14

### Example 2

Results

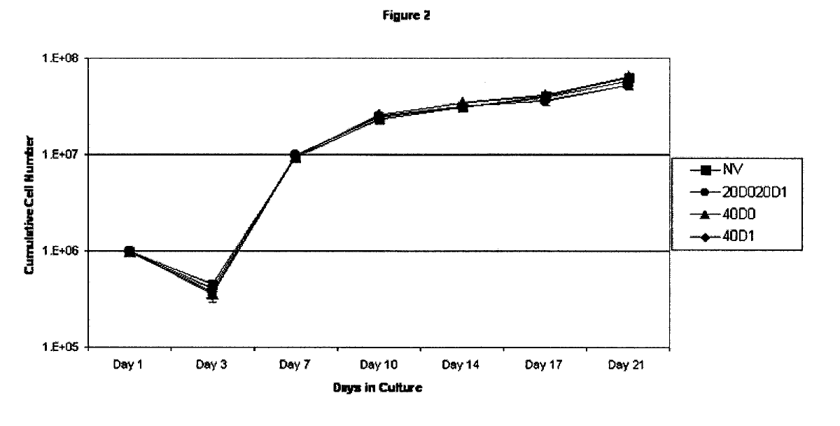

The effect of TAC on cell expansion was determined, and the results are shown in FIG. 2. On Day 21, expansion levels were close to 100-fold.

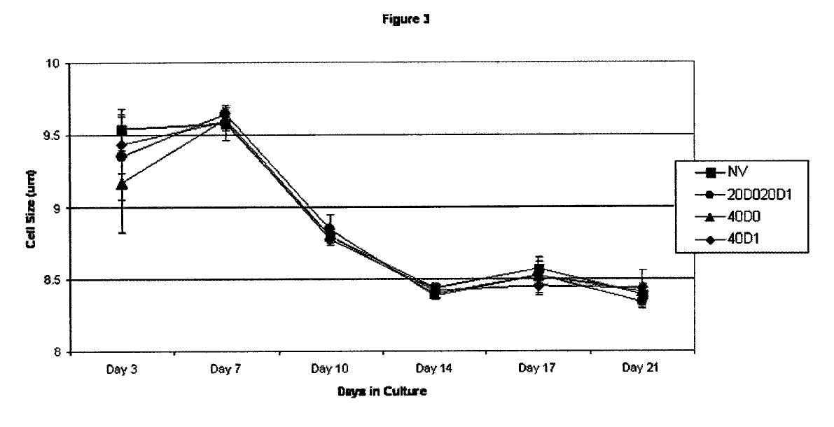

The effect of TAC on cell size was determined, and the results are shown in FIG. 3.

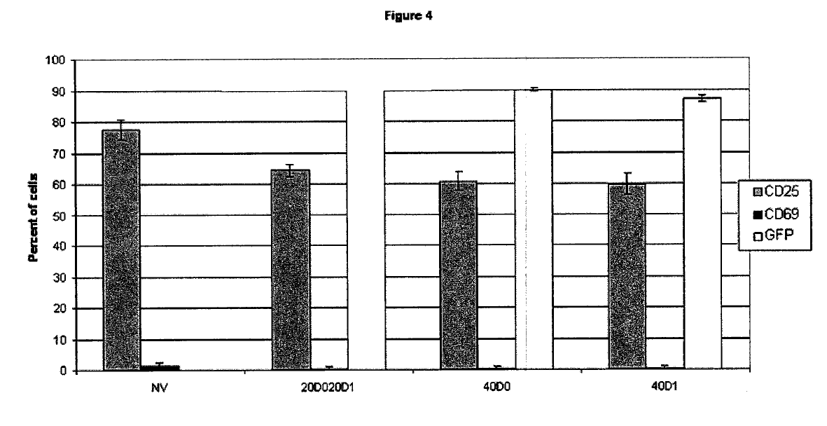

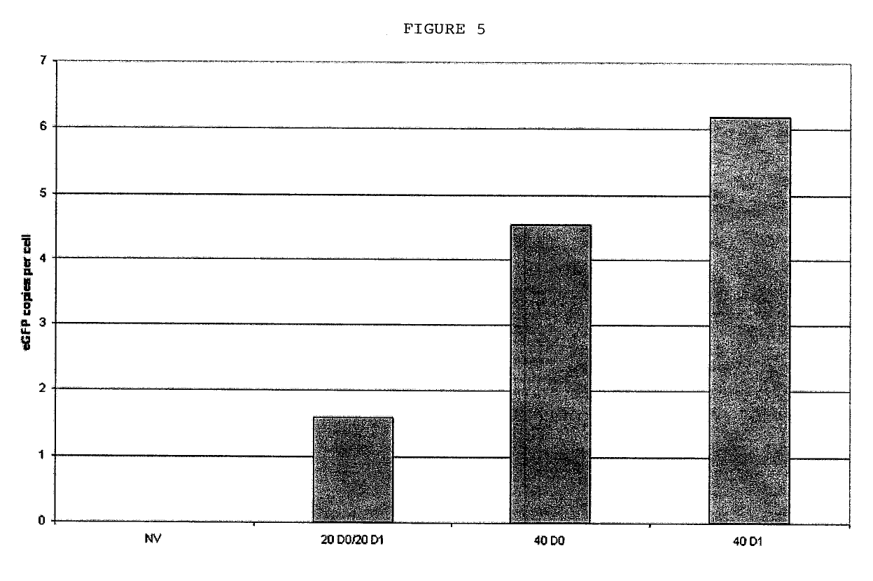

FACS analysis was used to detect the expression of eGFP, CD25, and CD69 in cells treated with TAC. The results are shown in FIG. 4. The data shows that TAC treated cells displayed excellent expression of the CD25 and CD69 markers. The TAC treated cells were transduced at levels reaching up to 92%.

Confirmation of transduction by vector was performed by quantitative PCR analysis (Taqman). The results are shown in FIG. 5.

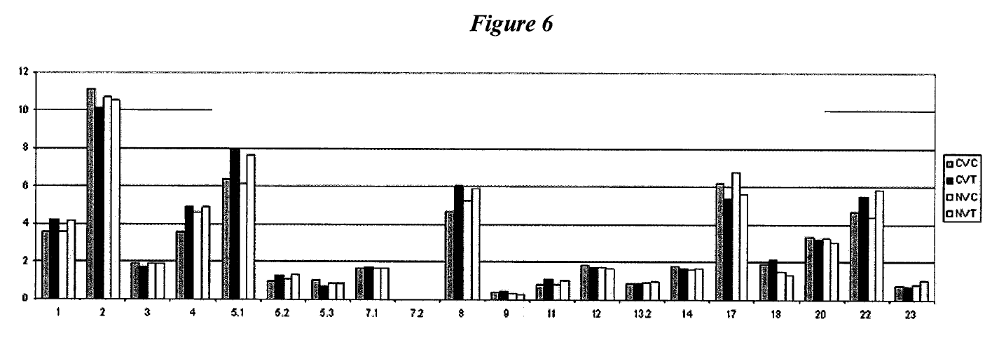

An experiment was conducted to determine the range and level of expression of several TCR Vβ families on cells that had been treated with TAC, and expanded for a period of 2 weeks. These data are presented in FIG. 6. Evident from the chart is that the range of TCR Vβ families expressed on expanded cells does not differ from the phenotype observed without TAC.

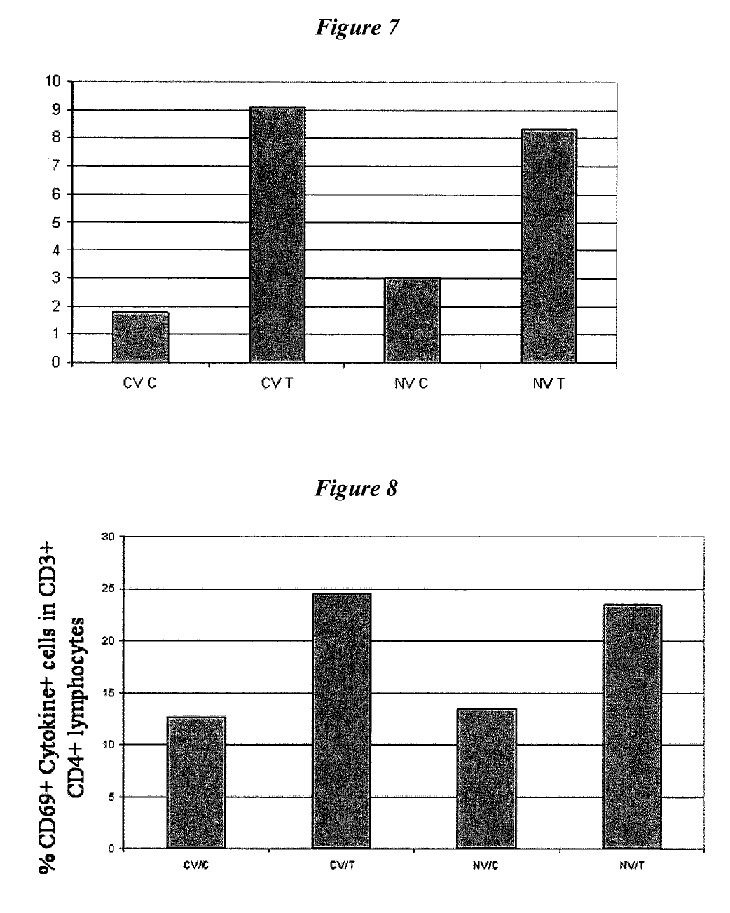

The ratio of live cells to dead cells was plotted after TAC treatment. The results are shown in FIG. 7, where the TAC treated cells had 5-fold fewer dead cells than control samples. Without being bound by theory, and offered to improve the understanding of the invention, a possible cause of these results could be that TAC does not over stimulate cells compared to other methods, where overstimulation leads to apoptosis.

FIG. 8 shows the intracellular cytokine staining (ICS) results after cells were stimulated with superantigen SEB (to stimulate cytokine production) for 6 hours. Non-SEB stimulated TAC-activated cells had low basal (or spontaneous) levels of cytokine production, but achieved high levels after SEB stimulation.

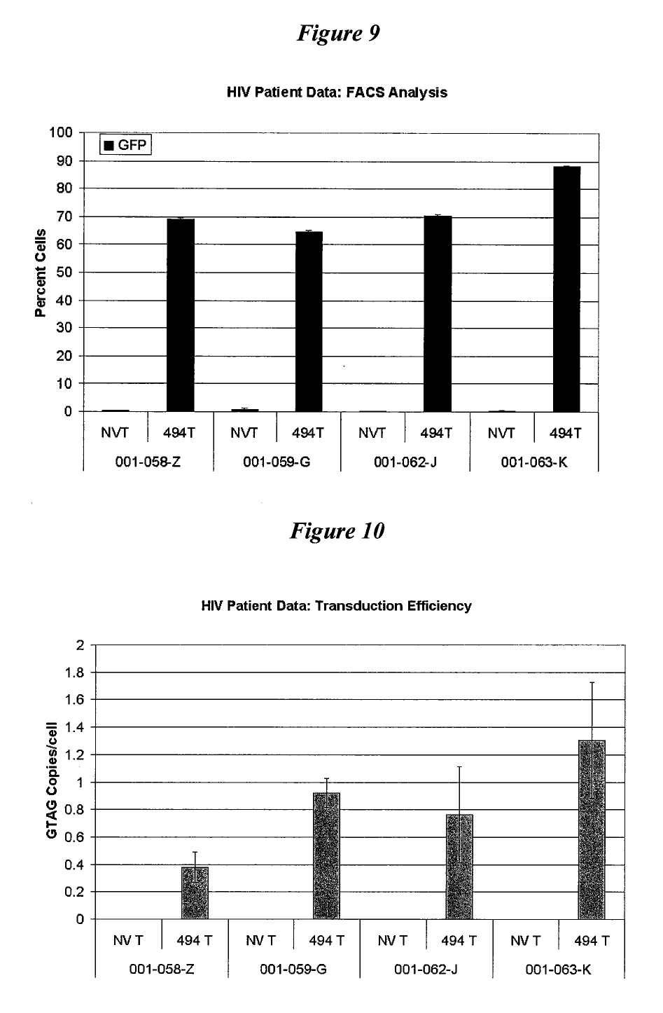

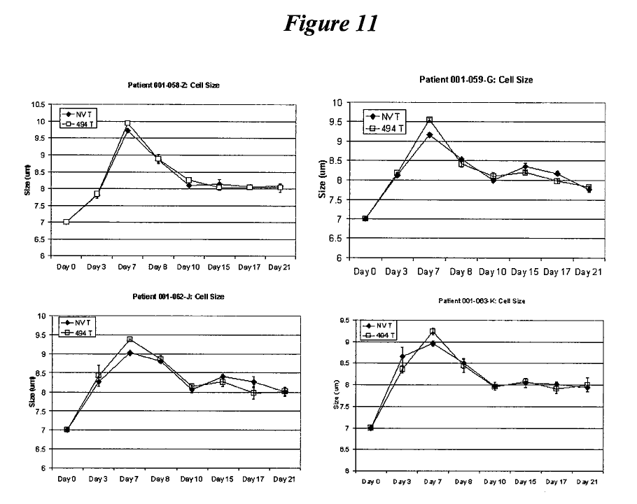

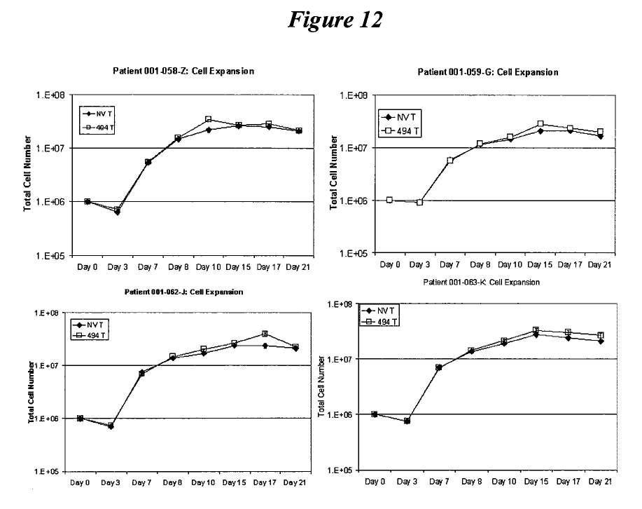

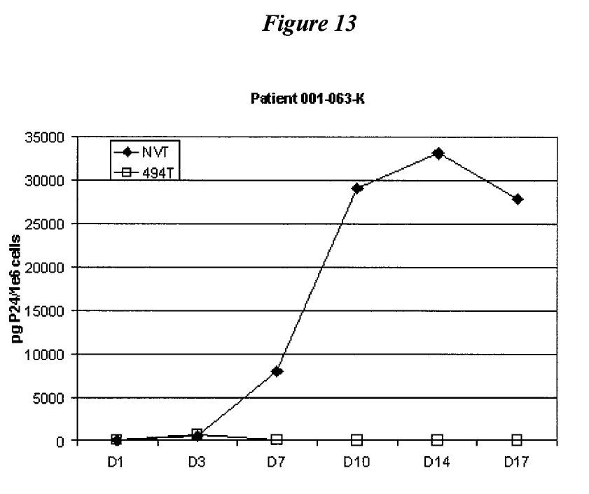

Historically, efficient transduction and expansion of cells isolated from HIV infected individuals was more difficult than with cells isolated from healthy individuals. To determine if cells isolated from an HIV infected patient could be efficiently transduced and expanded by TAC, cells isolated from apheresis products of 4 HIV infected individuals were evaluated as described in Example 1. The vector used to transduce the cells was a vector expressing antisense against HIV, so the biological activity of the vector could also be assessed by measuring production of the HIV capsid protein (p24) in culture supernatants, which is a well-accepted surrogate marker for HIV replication in vitro. FIGS. 9 and 10 show transduction efficiencies of these cells by measuring percent GFP positivity and copy number. FIGS. 11 and 12 present efficient cell activation (by size) and expansion. Finally, FIG. 13 presents the inhibition of endogenous HIV by transduced and untransduced cells. A time course is shown for patient 63.

Conclusion:

Altogether, the above described results indicate that the TAC treatment is a mild stimulation of T-cells in terms of causing cell death, and retains significant effect on transduction levels and expansion rates. The treatment also does not skew the population of expanded cells with respect to the TCR Vβ repertoire. Thus overall, the cells are likely to be healthy and may fare well upon introduction into a subject, such as when used as part of ex vivo therapy.

Importantly, data from cells isolated from HIV patients show that TAC continues to allow efficient transduction and expansion in this population, without compromising the anti-HIV potency of the treatment. This demonstrates the feasibility of this approach for ex vivo expansion of cells for preparation of a medicament for treatment of HIV, and potentially for other medicaments for human disease such as cancer.

All references cited herein, including patents, patent applications, and publications, are hereby incorporated by reference in their entireties, whether previously specifically incorporated or not.

Having now fully described this invention, it will be appreciated by those skilled in the art that the same can be performed within a wide range of equivalent parameters, concentrations, and conditions without departing from the spirit and scope of the invention and without undue experimentation.

While this invention has been described in connection with specific embodiments thereof, it will be understood that it is capable of further modifications. This application is intended to cover any variations, uses, or adaptations of the invention following, in general, the principles of the invention and including such departures from the present disclosure as come within known or customary practice within the art to which the invention pertains and as may be applied to the essential features hereinbefore set forth.

What is claimed is:

1. A soluble complex comprising a first ligand and a second ligand which binds a first and a second cell surface molecule, respectively, on the same target hemopoietic cell.

2. The complex according to claim 1 wherein each of said first and second ligands is an antibody or a fragment thereof which binds said first and second cell surface molecules, respectively.

3. The complex according to claim 1 wherein said target cell is a T cell.

4. The complex according to claim 1 wherein the first ligand binds CD28.

5. The complex according to claim 4 wherein the second ligand binds a ligand selected from B7-H1 (also called PD-L1); B7-H2; B7-H3 (also called B7RP-2); B7-H4; CD2; CD3; CD11a; CD26; CD27; CD30L; CD32; CD38; CD40L (also called CD154); CD45; CD49; CD50 (also called ICAM-3); CD54 (also called ICAM-1); CD58 (also called LFA-3); CD70; CD80 (also called B7.1); CD86 (also called B7.2); CD100; CD122; CD137L (also called 4-1BB Ligand); CD153; CTLA-4; ICOS; OX40L (also called CD134); PD-1; PD-L2 (also called B7-DC); SLAM (also called CD150); TIM-1; TIM-2; TIM-3; TIM-4; 2B4 (also called CD244); CD28; CD7; ICOS-L; ICAM; CD40; CD83; HLA-G; MICA; MICB; HVEM; lymphotoxin beta receptor; ILT3; ILT4; 3/TR6; 4-IBB; OX40; CD30; CD40; ICOS; LFA-1; CD7; LIGHT; NKG2C; BTLA; a Toll ligand receptor; or CD83.

6. The complex according to claim 1 wherein said first and second ligands are covalently linked or linked by one or more antibodies that bind both.

7. The complex according to claim 6 wherein said one or more antibodies are bi-specific antibodies, each of which bind both said first and second ligands.

8. The complex according to claim 1 wherein at least one of said first and second cell surface molecules is a transmembrane molecule.

9. The complex according to claim 1 wherein said first and second cell surface molecules are individually selected from B7-H1 (also called PD-L1); B7-H2; B7-H3 (also called B7RP-2); B7-H4; CD2; CD3 or CD3/TCR complex; CD11a; CD26; CD27; CD28; CD30L; CD32; CD38; CD40L (also called CD154); CD45; CD49; CD50 (also called ICAM-3); CD54 (also called ICAM-1); CD58 (also called LFA-3); CD70; CD80 (also called B7.1); CD86 (also called B7.2); CD100; CD122; CD137L (also called 4-1BB Ligand); CD153; CTLA-4; ICOS; OX40L (also called CD134); PD-1; PD-L2 (also called B7-DC); SLAM (also called CD150); TIM-1; TIM-2; TIM-3; TIM-4; or 2B4 (also called CD244).

10. A method of activating a hemopoietic cell, said method comprising
contacting said cell with a complex according to claim 1, wherein said contacting is optionally in vitro or ex vivo.

11. The method of claim 10 wherein said cell is a T cell.

12. A method of transducing a hemopoietic cell, said method comprising
introducing a nucleic acid molecule into said cell before, while, or after said cell is contacted with a complex according to claim 1, wherein said introducing is optionally in vitro or ex vivo.

13. The method of claim 12 wherein said cell is a T cell.

14. The method of claim 12 wherein said nucleic acid molecule is a viral vector.

15. A method of directing an enveloped vector to a target hemopoietic cell, said method comprising
contacting said target cell, optionally a T cell, with a combination of said enveloped vector and a complex according to claim 1 to form a vector-complex combination,
wherein said complex comprises a first ligand which binds a cell surface molecule in the envelope of said vector and a second ligand which binds a cell surface molecule of said target cell.

16. A method of directing a vector particle to a target hemopoietic cell, said method comprising
contacting said target cell, optionally a T cell, with a combination of said vector particle and a complex according to claim 1 to form a vector particle-complex combination,
wherein said complex comprises a first ligand which binds said particle and a second ligand which binds a cell surface molecule of said target cell.

17. The method of claim 14 wherein said viral vector is selected from a retroviral or lentiviral vector, optionally derived from avian reticuloendotheliosis virus (duck infectious anaemia virus, spleen necrosis virus, Twiehaus-strain reticuloendotheliosis virus, C-type retrovirus, reticuloendotheliosis virus Hungary-2 (REV-H-2)), and feline leukemia virus (FeLV)), human immunodeficiency viruses (HIV-1 and HIV-2), feline immunodeficiency virus (FIV), simian immunodeficiency virus (SIV), Maedi/Visna virus, caprine arthritis/encephalitis virus, equine infectious anaemia virus (EIAV), and bovine immunodeficiency virus (BIV); avian type C retroviruses, such as the avian leukosis virus (ALV); HTLV-BLV retroviruses, such as bovine leukaemia virus (BLV), human T-cell lymphotropic virus (HTLV), and simian T-cell lymphotropic virus; mammalian type B retroviruses, such as the mouse mammary tumor virus (MMTV); mammalian type C retroviruses, such as the murine leukaemia virus (MLV), feline sarcoma virus (FeSV), murine sarcoma virus, Gibbon ape leukemia virus, guinea pig type C virus, porcine type C virus, wooly monkey sarcoma virus, and viper retrovirus; spumavirus (foamy virus group), such as human spumavirus (HSRV), feline synctium-forming virus (FeSFV), human foamy virus, simian foamy virus, and bovine syncytial virus; and type D retroviruses, such as Mason-Pfizer monkey virus (MPMV), squirrel monkey retrovirus, and langur monkey virus.

18. The method of claim 17 wherein said viral vector is pseudotyped.

19. A method for ex vivo expansion of cells in the preparation of a medicament, said method comprising cell isolation and expansion after contact with the complex of claim 1.

20. The method of claim 19 where the cells are genetically modified, optionally with a retroviral vector.
